(19) United States
(12) Patent Application Publication
(10) Pub. No.: US 2015/0104857 A1
(43) Pub. Date: Apr. 16, 2015

(54) PHOTOBIOREACTOR SYSTEM FOR AIR PURIFICATION

(71) Applicant: Nano and Advanced Materials Institute Limited, Hong Kong (HK)

(72) Inventors: Di ZHANG, HONG KONG (HK); Yeuk Tin LAU, HONG KONG (HK); Ka Yip FUNG, HONG KONG (HK); Ka Ming NG, HONG KONG (HK)

(21) Appl. No.: 14/512,456

(22) Filed: Oct. 12, 2014

Related U.S. Application Data
(60) Provisional application No. 61/961,447, filed on Oct. 15, 2013.

(51) Int. Cl. C12M 1/00; C12M 1/107; B01D 53/84; C12N 1/12

(52) U.S. Cl. CPC C12M 21/02; C12N 1/12; C12M 29/06; C12M 23/36; C12M 41/14; B01D 53/84; B01D 2257/504; B01D 2251/95
USPC 435/266; 435/292.1

(57) ABSTRACT

The present invention relates to air purification. The present invention provides a system for reducing carbon dioxide concentration in air in locations with sub-tropical to temperate climates and method of reducing carbon dioxide concentration using the system.

### CROSS REFERENCE TO RELATED APPLICATION

Pursuant to 35 U.S.C. §119(e), this is a non-provisional patent application claiming benefit from U.S. provisional patent application Ser. No. 61/961,447 filed Oct. 15, 2013, and the disclosure of which is incorporated herein by reference in its entirety.

### FIELD OF THE INVENTION

The present invention relates to air purification. In particular, the present invention relates to a system for reducing carbon dioxide concentration in air using photosynthetic organism. The present invention is particularly suitable for use in places of sub-tropical and temperate climates.

### BACKGROUND

Emission of greenhouse gases from vehicles and industries is one of the major causative factors for global warming and leads to temperature increase on earth. Carbon dioxide (CO₂) accounts for 68% of the greenhouse gases. CO₂ sequestration by various methods has been widely studied, aims to reduce the emission of CO₂ into the atmosphere. Conventionally, CO₂ in air is captured by chemical and physical technologies, such as scrubbing with aqueous amine solutions together with solvent regeneration at high temperature and physical solvent separation using high pressure, etc. The captured CO₂ is then transported to offshore geological storage (Gibbins et al., 2008).

Biological method for air purification is now being considered as a better alternative as compared to the conventional chemical and physical methods. Biological method requires milder conditions. High temperature and pressure, and environmental harmful solvents used in conventional methods are excluded in biological method. Biological method is also more sustainable. Biological method relies on photosynthesis to convert unwanted CO₂ to oxygen, thus physical storage space for storing CO₂ is not needed. Additional to oxygen, valuable biomass (e.g. biodiesel) and chemicals which are useful for fish feed and fertilizer are produced during removal of CO₂ by biological methods (Chisti, 2007).

However, local climate affects performance of the photobioreactor in removing carbon dioxide. Therefore, it is desired to provide a photobioreactor system adapted to the local climate and environment factors for efficient removal of carbon dioxide in air.

### SUMMARY OF THE INVENTION

Among photosynthetic biomass organisms, microalgae are regarded to be the most suitable photosynthetic organisms for the application of air purification photobioreactor system, due to its high photosynthetic efficiency (Perrine et al., 2012).

Accordingly, the present invention provides a system for air purification specific for locations with sub-tropical to temperate climate using microalgae.

A first aspect of the present invention provides a photobioreactor system for reducing carbon dioxide in air in a location with sub-tropical to temperate climate comprises:

a reactor tank for housing a culture medium comprising Chlorella species at a concentration of 1,000,000-1,500,000 cells/mL at a predetermined temperature of 5-40° C.;

a gas inlet where the air enters into the system;

a gas pump and a sparger for feeding the air to the culture medium at a flow rate of 0.1-2.0 L/min as small bubbles;

a temperature control to regulate temperature of the reactor tank such that temperature of the air and the culture medium are maintained at the predetermined temperature;

a light source for providing light to the culture medium of an intensity of 50-500 μmolm⁻²s⁻¹PPFD; and

a gas outlet where purified gas having a reduced carbon dioxide concentration exits the system.

In accordance with an embodiment of the presently claimed invention, the culture medium further comprises Nannochloropisis salina.

In accordance with an embodiment of the presently claimed invention, the location with sub-tropical to temperate climate is Southeast Asia.

In accordance with another embodiment of the presently claimed invention, the location is Hong Kong.

In accordance with yet another embodiment of the presently claimed invention, the culture medium and the air are maintained at the predetermined temperature of 15-30° C.

In accordance with another embodiment of the presently claimed invention, the culture medium is maintained at pH 7-9.

In accordance with another embodiment of the presently claimed invention, said light source comprises light emitting diode, sunlight or both.

A second aspect of the presently claimed invention is to provide a method for reducing carbon dioxide in air in a location with sub-tropical to temperate climate comprises:
providing a bioreactor system;
feeding the air into the bioreactor system via a gas inlet;
adjusting temperature of the bioreactor system at the predetermined temperature such that the air and the culture medium is maintained at the predetermined temperature;
providing light to the culture medium of an intensity of 50-500 μmolm⁻²s⁻¹PPFD; and
feeding the air at the predetermined temperature to the culture medium at a flow rate of 0.1 to 2.0 L/min as small bubbles.

Unlike any existing bioreactor, the present photobioreactor system and method of air purification using the same are adapted to sub-tropical to temperate climates for high efficient removal of carbon dioxide.

### BRIEF DESCRIPTION OF THE DRAWINGS

Embodiments of the present invention are described in more detail hereinafter with reference to the drawings, in which:

FIG. 1 is a schematic diagram of a panel shaped photobioreactor of the present invention;

FIG. 2 is a schematic diagram of a tubular shaped photobioreactor of the present invention.

FIG. 3 are graphs showing specific growth rate of Chlorella species at different light intensities (FIG. 3A) and different temperatures (FIG. 3B).

### DETAILED DESCRIPTION OF PREFERRED EMBODIMENTS

To illustrate the structure and advantages of the present invention, below is the detailed description of the present invention in combination with the figures and embodiments.

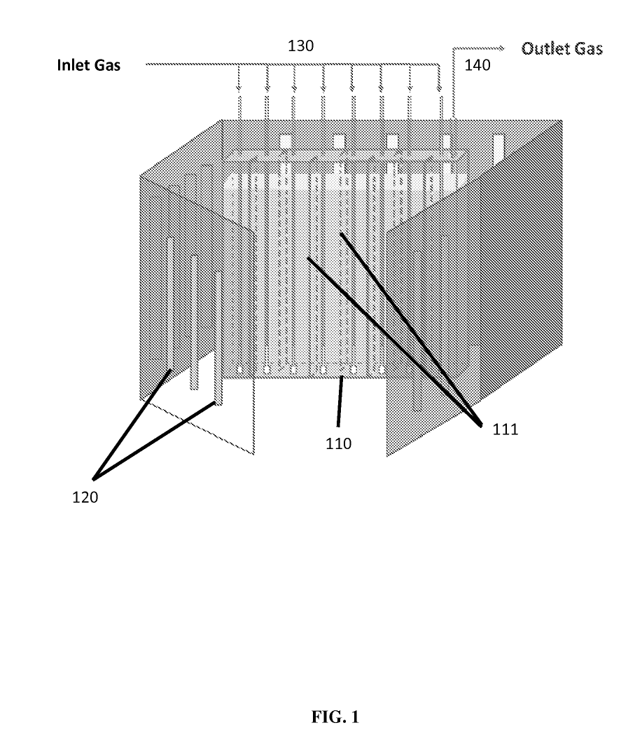

As shown in FIG. 1, the photobioreactor system of the present invention comprises a flat panel shaped reactor tank 110 for housing culture medium. The reactor tank of the present invention may be transparent, translucent or reflective such that light can pass through the reactor tank and reaches to the microalgae housed therein for photosynthesis. The culture medium comprises Chlorella species at a concentration of 1,000,000-1,500,000 cells/mL. Chlorella species applicable for the present invention includes, but are not limited to, Chlorella pyrenoidosa and Chlorella vulgaris. In one embodiment of the presently claimed invention, the culture medium further comprises Nannochloropisis salina. Specifically, it is found by inventors of the present application that cultures of Chlorella species or together with N. salina exert long and stable lifespan in locations with sub-tropical to temperate climate to allow efficient removal of carbon dioxide to take place. It is found that addition of species other than Chlorella and N. salina to the culture lead to shorter and less stable lifespan that fail to provide efficient carbon dioxide removal. In one embodiment, the culture medium comprises no more than two types of microalgae species. In another embodiment, one of the two types of microalgae species is Chlorella species. In yet another embodiment, the second type of microalgae is N. salina.

The reactor tank may or may not be partitioned by a plurality of partitions 111 to arrange various internal flow patterns. For instance, the plurality of partitions 111 consist of partitions projecting from opposite sides of the reactor tank alternatively. The photobioreactor system of the present invention further comprises one or more gas inlet 130 for feeding the air being treated into the culture medium. The air being treated is fed into the culture medium as bubbles by sparger or any other applicable means readily used in the art. In one embodiment, the air being treated bubbles into the culture medium at the bottom of the reactor tank at a flow rate of 0.1-2.0 L/min. The temperature of the culture medium is controlled at 5-40° C. A water bath or any other temperature control system in the art may be used in the present system to control the temperature of the reactor tank so as to control the temperature of the culture medium and the air being treated. In one embodiment, the temperature of the reactor tank, culture medium and the air being treated are being maintained at 5-40° C., or at 15-30° C. In another embodiment, the temperature of the culture medium and the air being treated are the same. The present photobioreactor system includes a temperature control (not shown in FIG. 1) that maintains the temperature of the reaction tank within a predetermined temperature range. The reaction tank is maintained at 5-40° C., or at 15-30° C. By controlling the temperature of the reaction tank, the temperature of the air being treated is also being kept at a temperature equal to the reaction tank and the culture medium. If the air temperature is different from temperature of the culture medium, the temperature control is also capable of adjusting the temperature inside the reactor tank in order to maintain the temperature of the culture medium and the air being treated at the predetermined temperature. The medium for culturing the microalgae Chlorella species or Chlorella species together with N. salina may be any type of medium suitable for growth of the microalgae. A mixture of medium may also be used. In one embodiment, the medium is Bold's basal medium. In another embodiment, the medium is a mixture of Bristol medium and the Trace medium. Below shows the chemical composition and preparation of the Bristol medium and the Trace medium for use in the present invention.

Bristol Medium

### Bristol A

| Chemical | Stock | |
|---|---|---|
| Sodium Nitrate (NaNO₃) | 25 | gram/L |
| Dipotassium Phosphate (K₂HPO₄) | 7.5 | gram/L |
| Monopotassium Phosphate (KH₂PO₄) | 17.5 | gram/L |
| Sodium Chloride (NaCl) | 2.5 | gram/L |

### Bristol B

| Chemical | Stock |
|---|---|
| Calcium Chloride (CaCl₂) | 2 gram/L |

### Bristol C

| Chemical | Stock |
|---|---|
| Magnesium Sulfate Heptahydrate (MgSO₄ · 7H₂0) | 7.5 gram/L |

100 fold dilution of components Bristol A, B and C as indicated above are combined to prepare the Bristol medium.

Trace Medium

### Trace 1 (Alkaline EDTA)

| Chemical Name | Stock |
|---|---|
| Etylenediaminetetraacetic acid (EDTA) | 50 gram/L |
| Potassium hydroxide (KOH) | 31 gram/L |

### Trace 2 (Trace Metal)

| Chemical Name | Stock |
|---|---|
| Zinc Sulfate Heptahydrate (ZnSO₄ · 7H₂0) | 8.82 gram/L |
| Manganese(II) Chloride Tetrahydrate (MnCl₂ · 4H₂O) | 1.44 gram/L |
| Sodium Molybdate (Na₂MoO₄) | 1.19 gram/L |
| Copper(II) Sulfate Pentahydrate (CuSO₄ · 5H₂0) | 1.57 gram/L |
| Cobalt(II) Chloride Hexahydrate (CoCl₂ · 6H₂0) | 0.40 gram/L |

### Trace 3 (Acidified Acid)

| Chemical Name | Stock |
|---|---|
| Iron(II) Sulfate Heptahydrate (FeSO₄ · 7H₂O) | 4.98 gram/L |
| Sulfuric Acid (H₂SO₄) | 1 miliLitre/L |

### Trace 4 (Boric Acid)

| Chemical Name | Stock |
|---|---|
| Boric Acid (H₃BO₃) | 11.42 gram/L |

1000 fold dilution of components trace 1-4 are combined to prepare the Trace medium.

pH of the culture medium is also maintained at pH 7-9. When the present photobioreactor is operating continuously, density of the culture and pH may be maintained at 1,000,000-1,500,000 cells/mL by removing the microalgal biomass and replacing fresh culture medium in the reactor tank.

The photobioreactor system further comprises a gas outlet 140 where purified air with reduced carbon dioxide exits the reactor tank. In one embodiment, the gas inlet is positioned at upper portion of the photobioreactor. The gas being treated are bubbled into the culture medium at the bottom of the reactor tank and are purified by the microalgae culture such that purified air with reduced carbon dioxide rises to the top of the reactor tank and exits through the gas outlet. The Chlorella species or Chlorella species together with N. salina of the culture medium carries out photosynthesis with the carbon dioxide in the air being treated. The culture medium converts carbon dioxide into oxygen. Thus, the air after treatment with the culture medium is purified with reduced carbon dioxide.

The photobioreactor system of the present invention further comprises a light source for providing light to the culture medium for photosynthesis. In one embodiment, the light source is in a form of a light chamber 120 having an array of LED lights situated around the outside of the reactor tank 110. Fluorescent lamps, high-pressure sodium map, fluorescent mercury lamp, sunlamp, or sunlight may be used. In one embodiment, a light intensity of 50-500 μmolm⁻²s⁻¹PPFD is supplied to the culture medium for efficient removal of carbon dioxide. In an exemplary embodiment, the array of LED lights or other light source may be situated in a position perpendicular to the orientation of the plurality of the partitions 111 such that the photosynthetic rate can be increased by maximizing the light source reaching to the culture medium inside the reactor tank 110.

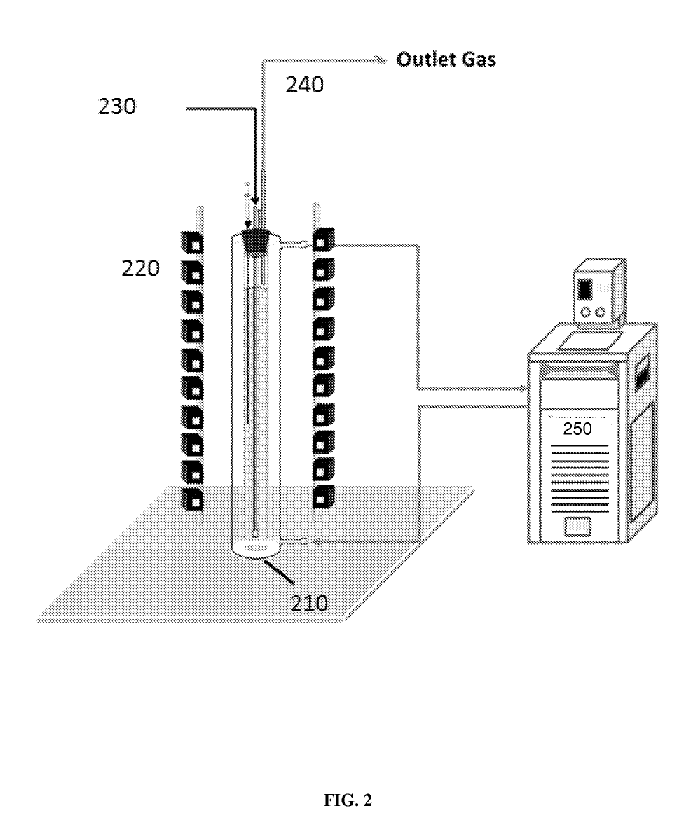

FIG. 2 illustrates another embodiment of the present photobioreactor in which reactor tank 210 is tubular in shape. A vertically arranged array of LEDs 220 positioned along the tubular reactor tank, a gas inlet 230 extended to the bottom of the reactor tank to feed air into the culture medium and a gas outlet 240 at the top of the reactor tank are shown. The photobioreactor system further comprises sensors to monitor temperature, gas flow rate, light intensity and pH in the reactor tank and corresponding controlling means to maintain the desirable ranges of temperature, light intensity, flow rate and pH for efficient carbon dioxide removal in sub-tropical to temperate climate. In this embodiment, a temperature controller 250 is used to monitor the air temperature and adjust the same to the predetermined temperature.

The presently claimed invention also provides a method for reducing carbon dioxide in air in a location with sub-tropical to temperate climate comprises providing a photobioreactor system in accordance with the present invention, feeding the air into the bioreactor system, adjusting temperature of reactor tank of the photobioreactor system so as to maintain the temperature of the air and the culture medium at a predetermined temperature of 5-40° C. The method of reducing carbon dioxide for purification of the present invention comprises providing the culture medium at 1,000,000-1,500,000 cells/mL, adjusting temperature of the reactor tank at the predetermined temperature of 5-40° C. such that the temperature of the incoming gas being treated and the culture medium are being maintained at the predetermined temperature, feeding the incoming gas being treated into the culture medium at 0.1 to 2.0 L/min, enabling purified gas having reduced concentration of carbon dioxide than the incoming gas to exit the photobioreactor system and optionally collecting any biomass produced during photosynthesis of the culture medium.

The present photobioreactor system demonstrates highly efficient carbon dioxide purification in locations with sub-tropical to temperate climate. The culture, culture density, temperature, flow rate and light intensity are selected for high efficiency carbon dioxide purification in locations with sub-tropical to temperate climates. The locations have hot, humid summers and generally mild winters. In particular, the location has a mean temperature between −3° C. and 22° C. The present photobioreactor system is designed for high efficiency carbon dioxide purification in Southeast Asia. The present photobioreactor system is designed for high efficiency carbon dioxide purification in Hong Kong or location with similar climate.

Examples are given below to demonstrate the operation of the present invention for air purification in Hong Kong using microalgae which can significantly reduce the CO₂ concentration in air.

### EXAMPLE 1

Purification of CO₂ polluted air using the present invention as shown in FIG. 1 is carried out. Chlorella sp. in modified Bold's Basal medium is maintained at 1,200,000 cells of microalgae/mL medium. The temperature of the reactor tank is 25° C. A LED setup radiates light at 200 μmol/m²s⁻¹. The CO₂ concentration of the inlet gas is 11.5% and its flow rate is 600 mL/min. After 24 hours, the concentration of the CO₂ in the outlet gas is measured to be 3.5%.

### EXAMPLE 2

Purification of CO₂ polluted air using the present invention as shown in FIG. 2 is carried out. Chlorella sp. in modified Bold's Basal medium is maintained at 1,200,000 cells of microalgae/mL medium. The temperature of the reactor tank is 20° C. A LED setup radiates light at 80 μmol/m²m⁻¹. The CO₂ concentration of the inlet gas is 500 ppm and its flow rate is 350 mL/min. After 5 days, the concentration of the CO₂ in the outlet gas is measured to be 130 ppm.

### EXAMPLE 3

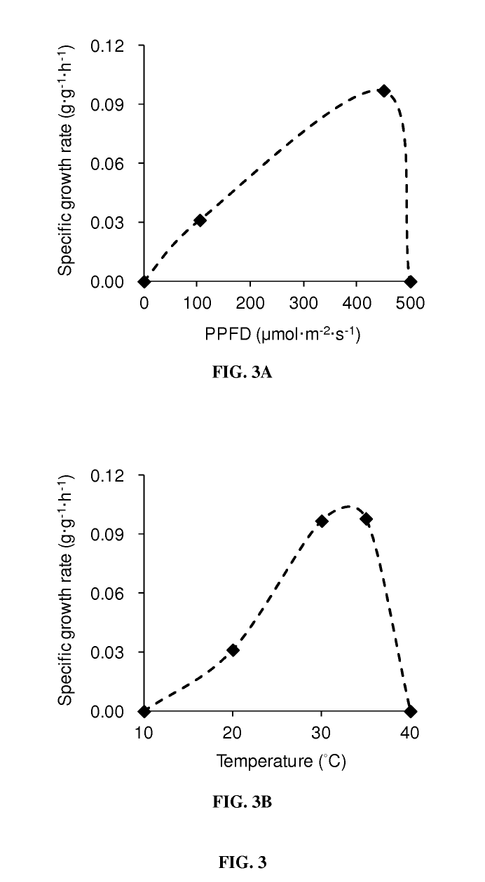

The effects of temperature and light intensity on growth rate of the Chlorella species are investigated. The specific growth rate of Chlorella species at different light intensities expressed as photosynthetic photon flux density (PPFD) at 20° C. and 7.5% v/v carbon dioxide concentration is measured (FIG. 3A). It is shown that growth of Chlorella species increases with increase of light intensity up to approximately 450 μmolm⁻²s⁻¹. The growth of Chlorella species decreases abruptly at light intensity above 450 μmolm⁻²s⁻¹. The specific growth rate of Chlorella species at different temperatures under constant 7.5% v/v of carbon dioxide concentration and at light intensity of 105 μmolm⁻²s⁻¹ is then studied. As seen in FIG. 3B, Chlorella growth is higher at a higher temperature up to 35° C., after that it decreases sharply at temperature higher than 35° C. These results show that the temperature and light intensity ranges required in the present photobioreactor system promotes optimal microalgae growth for photosynthetic carbon dioxide removal.

The foregoing description of the present invention has been provided for the purposes of illustration and description. It is not intended to be exhaustive or to limit the invention to the precise forms disclosed. Many modifications and variations will be apparent to the practitioner skilled in the art.

The embodiments were chosen and described in order to best explain the principles of the invention and its practical application, thereby enabling others skilled in the art to understand the invention for various embodiments and with various modifications that are suited to the particular use contemplated. It is intended that the scope of the invention be defined by the following claims and their equivalence.

1. A system for reducing carbon dioxide in air in a location with sub-tropical to temperate climate comprising
a reactor tank for housing a culture medium comprising Chlorella species at a concentration of 1,000,000-1,500,000 cells/mL at a predetermined temperature of 5-40° C.;
a gas inlet where the air enters into the system;
a gas pump and a sparger for feeding the air to the culture medium at a flow rate of 0.1-2.0 L/min as small bubbles;
a temperature control to regulate temperature of the reactor tank such that temperature of the air and the culture medium are maintained at the predetermined temperature;
a light source for providing light to the culture medium of an intensity of 50-500 μmolm⁻¹s⁻¹PPFD; and
a gas outlet where purified gas having a reduced carbon dioxide concentration exits the system.

2. The system of claim 1, wherein the culture medium further comprises Nannochloropoisis salina.

3. The system of claim 1, wherein the location is Southeast Asia.

4. The system of claim 1, wherein the location is Hong Kong.

5. The system of claim 1, wherein the Chlorella species comprises Chlorella pyrenoidosa, Chlorella vulgaris or combination thereof.

6. The system of claim 1, wherein the culture medium comprises Bold's basal medium or a mixture of Bristol medium and Trace medium.

7. The system of claim 6, wherein the Bristol medium comprises sodium nitrate, dipotassium phosphate, monopotassium phosphate, sodium chloride, calcium chloride and magnesium sulfate heptahydrate.

8. The system of claim 6, wherein the Trace medium comprises etylenediaminetetraacetic acid, potassium hydroxide, zinc sulfate heptahydrate, manganese(II) chloride tetrahydrate, sodium molybdate, copper(II) sulfate pentahydrate, cobalt(II) chloride hexahydrate, iron(II) sulfate heptahydrate and sulfuric acid

9. A method for reducing carbon dioxide in air in a location with sub-tropical to temperate climate, said method comprises:
providing a system of claim 1;
feeding the air into the system via the gas inlet;
adjusting temperature of the reactor tank to the predetermined temperature such that temperature of the air and the culture medium are maintained at the predetermined temperature;
providing light to the culture medium of an intensity of 50-500 μmolm⁻²s⁻¹PPFD; and
feeding the air at the predetermined temperature to the culture medium at a flow rate of 0.1 to 2.0 L/min as small bubbles.

10. The method of claim 9, wherein the culture medium further comprises Nannochloropoisis salina.

11. The method of claim 9, wherein the location is Southeast Asia.

12. The method of claim 9, wherein the location is Hong Kong.

13. The method of claim 9, wherein the Chlorella species comprises Chlorella pyrenoidosa, Chlorella vulgaris or combination thereof.

14. The method of claim 9, wherein the culture medium comprises Bold's basal medium or a mixture of Bristol medium and Trace medium.

15. The method of claim 14, wherein the Bristol medium comprises sodium nitrate, dipotassium phosphate, monopotassium phosphate, sodium chloride, calcium chloride and magnesium sulfate heptahydrate.

16. The method of claim 14, wherein the Trace medium comprises etylenediaminetetraacetic acid, potassium hydroxide, zinc sulfate heptahydrate, manganese(II) chloride tetrahydrate, sodium molybdate, copper(II) sulfate pentahydrate, cobalt(II) chloride hexahydrate, iron(II) sulfate heptahydrate and sulfuric acid
